(19) United States
(12) Patent Application Publication
(10) Pub. No.: US 2024/0180433 A1
(43) Pub. Date: Jun. 6, 2024

(54) SYSTEM AND METHOD FOR NON-INVASIVE HEALTH MONITORING

(71) Applicant: RAY IOT SOLUTIONS INC., Lewes, DE (US)

(72) Inventors: Ranjana Nair, Bangalore (IN); Sanchi Poovaya, Mysore (IN)

(73) Assignee: RAY IOT SOLUTIONS INC., Lewes, DE (US)

(21) Appl. No.: 18/438,555

(22) Filed: Feb. 12, 2024

Related U.S. Application Data
(63) Continuation of application No. 15/688,150, filed on Aug. 28, 2017.

(30) Foreign Application Priority Data
Aug. 26, 2016 (IN) 201641029076

(51) Int. Cl. A61B 5/0205; A61B 5/00; A61B 5/01; A61B 5/08; A61B 5/113; G16H 40/63; G16H 40/67; G16H 50/20

(52) U.S. Cl. CPC A61B 5/02055; A61B 5/0064; A61B 5/0803; A61B 5/0816; A61B 5/1135; G16H 40/63; G16H 40/67; G16H 50/20; A61B 5/01; A61B 5/72; A61B 2503/04

(57) ABSTRACT

Systems and methods for contactless monitoring of a patient's health. The system includes a monitoring device configured to monitor one or more health parameters of the patient. The monitoring device includes sensors configured to obtain signals pertaining to the health parameters without making physical contact with the patient. The system further includes a means to store data pertaining to the signals, a means to process and analyze the data, and a means to send an alert if the analysis indicates a patient health issue has occurred.

### CLAIM OF PRIORITY

This application claims priority to U.S. patent application Ser. No. 15/688,150 filed Aug. 8, 2017, which claims priority to Indian Patent Application No. 201641029076, filed Aug. 26, 2016, both of which are incorporated herein in their entirety.

### FIELD

The disclosed embodiments are generally related to health monitoring devices, and more particularly to non-invasive, non-contact health monitoring devices.

### BACKGROUND

Health monitoring systems are continuously being developed in recent years. More specifically, these devices continuously monitor the health of patients based on physiological parameters. The demand for health monitoring devices and systems is increasing due to the rapid development in this field of technology, increased health awareness, increasing health costs, aging population, and the like. To address this demand, a variety of prototypes and commercial products have been developed in the course of recent years which aim at providing real-time feedback information about the patient's health condition, either to the user himself or to a medical center or to a supervising professional physician, in case of possible health threatening conditions.

The conventional health monitoring systems are mostly invasive and/or require contact with the human body and may pose problems to the patients while using them. Even the conventional non-invasive health monitoring systems require the user to be in contact with the device such as a strip, sock, bed, etc., which may be uncomfortable to the user. Hence, there is a need for a system and method for non-invasive non-contact health monitoring. Further, there is a need for a system and method for alerting the patient and a caregiver using the non-contact non-invasive health monitoring system. Furthermore, there is a need for a method and system for monitoring and analyzing patient data using artificial intelligence.

The above-mentioned shortcomings, disadvantages, and problems are addressed herein and which will be understood by reading and studying the following specification.

### SUMMARY

Any electronic device that is in contact with an infant's body has a safety risk, as most battery powered devices have Li ion batteries. According to an aspect of the present invention, a non-contact non-invasive solution is presented that is capable of remotely collecting accurate respiration and motion data enables the running of artificial intelligence algorithms that will analyze the data and warn caregivers and emergency response teams of any irregularities. Without constant monitoring, there is a significant chance that users who are predisposed with this condition can have an attack that could be fatal. The present invention cuts the time that occurs between monitoring the patient to notifying the caregivers of potential life-threatening attacks/irregularities, and this is exceedingly crucial in saving lives.

According to an aspect of the present invention, a system for monitoring a patient's health is provided. The system includes a monitoring device configured to monitor one or more health parameters of the patient, wherein the monitoring device includes a sensing unit configured to receive the signals pertaining to the one or more health parameters, wherein the sensing unit includes one or more sensors configured to receive signals through non-physical contact with the patient. The system further includes a memory configured to store data pertaining to the signals, and a processor configured to analyze the data using pre-determined information and programmed artificial intelligence.

According to another aspect of the present invention, a method for monitoring a patient's health is provided. The method includes receiving, using a sensing unit coupled to a monitoring device, one or more signals pertaining to one or more health parameters of the patient, wherein the sensing unit includes one or more sensors, analyzing, using a processor, data pertaining to the one or more signals, the analyzing including determining one or more medical conditions of the patient using pre-determined information and programmed artificial intelligence, and transmitting, to a remote server, data analyzed using the processor.

It is an object of the present invention to provide the system for monitoring a patient's health, wherein the one or more health parameters are selected from the group consisting of: respiration patterns; heart rate; temperature; and sleep patterns.

It is an object of the present invention to provide the system for monitoring a patient's health, wherein the monitoring device is a wall- or table-mounted device.

It is an object of the present invention to provide the system for monitoring a patient's health, wherein the monitoring device is further configured to transmit and receive one or more electromagnetic signals.

It is an object of the present invention to provide the system for monitoring a patient's health, wherein the one or more sensors are selected from the group consisting of: an ultrasound reader (Ultra Wide Band (UWB) radar sensor); a camera (and IR sensor); a camera; a video recorder; and a microphone.

It is an object of the present invention to provide the system for monitoring a patient's health, wherein the system further includes a data transceiver configured to receive data from the sensing unit and transmit the data to a wireless server.

It is an object of the present invention to provide the system for monitoring a patient's health, wherein the system further includes a mobile electronic device configured to receive data analyzed by the processor.

It is an object of the present invention to provide the system for monitoring a patient's health, wherein the mobile electronic device further includes a graphical user interface configured to enable a user to access data stored in the memory.

It is an object of the present invention to provide the system for monitoring a patient's health, wherein the processor is further configured to send one or more alerts to the mobile electronic device if a pre-determined medical condition is met based on the analyzed data.

It is an object of the present invention to provide the method for monitoring a patient's health, wherein the analyzing further includes incorporating analytics, thresholding, filtering, noise reduction, digital signal processing algorithms, and artificial intelligence algorithms.

It is an object of the present invention to provide the method for monitoring a patient's health, wherein the one or more health parameters are selected from the group consisting of: respiration patterns; and sleep patterns.

It is an object of the present invention to provide the method for monitoring a patient's health, wherein the monitoring device is a wall (or table) mounted device.

It is an object of the present invention to provide the method for monitoring a patient's health, wherein the one or more sensors are selected from the group consisting of: an ultra-wide band radar wave sensor; an air quality sensor; an ultrasound reader; a camera; a video recorder; and a microphone.

It is an object of the present invention to provide the method for monitoring a patient's health, wherein the method further includes transmitting, to a mobile electronic device or a network of devices, data analyzed by the processor.

It is an object of the present invention to provide the method for monitoring a patient's health, wherein the method further includes transmitting, to a mobile electronic device, data analyzed in the cloud.

It is an object of the present invention to provide the method for monitoring a patient's health, wherein the method further includes accessing the data analyzed by the processor, using a graphical user interface coupled to the mobile electronic device.

It is an object of the present invention to provide the method for monitoring a patient's health, wherein the method further includes sending an alert to a user if a pre-determined medical condition is met based on the data analyzed by the processor.

### BRIEF DESCRIPTION OF THE DRAWINGS

The other objects, features, and advantages will occur to those skilled in the art from the following description of the preferred embodiment and the accompanying drawings in which:

FIG. 1 is a flow chart of an exemplary method of signal processing in accordance with the disclosure.

FIG. 2 shows a system diagram illustrating a non-invasive, non-contact health monitoring device, in accordance with the disclosure.

FIG. 3 shows a perspective view of a device for providing a non-invasive, non-contact health monitoring device, in accordance with the disclosure.

FIG. 4 shows a sectional side view of the health monitoring device of FIG. 3, in accordance with the disclosure.

FIGS. 5A and 5B show 3D graphs of a raw signal input to a health monitoring device and a filtered signal output, respectively, in accordance with the disclosure.

FIGS. 6A and 6B show a non-contact health monitoring environment and device, respectively, in accordance with the disclosure.

Although certain features of the present invention may be shown in some drawings and not in others, this is done for convenience only, as each feature may be combined with any or all of the other features in accordance with the present invention.

### DETAILED DESCRIPTION

Exemplary embodiments of the present invention will now be described with reference to the drawings, in which identical elements in the various figures are identified with the same reference numerals.

Reference will now be made in detail to exemplary embodiments of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

The various embodiments of the present invention provide systems and methods for continuously monitoring the health of a user. The system includes a monitoring device that monitors the health parameters of patients using respiration, body movement, and sleep patterns. According to an embodiment, the monitoring device monitors heart rate, temperature, and/or any other suitable biometric data. According to an embodiment of the present invention, the monitoring device is a wall (or table) mounted device that is capable of transmission and reception of electromagnetic signals. The wall (or table) mounted device includes a sensing unit to receive a respiration and motion signals of the patients under observation. According to an embodiment, the sensing unit includes one or more sensors. According to an embodiment, a sensing unit, located in the wall (or table) mounted device, senses the respiration and motion of the patient under observation and analyzes respiration and motion patterns using signal processing algorithms and artificial intelligence algorithms.

According to an embodiment, the data collected from the sensing unit is continuously uploaded to the cloud. According to an embodiment, the data collected from the sensing device is processed in two ways. The processing unit on the wall (or table) mounted device processes the data from the sensing unit using filtering algorithms, thresholding algorithms, noise reduction, digital signal processing algorithms, and/or artificial intelligence algorithms. The cloud is also configured to support processing of data, more specifically, thresholding and artificial intelligence algorithms. The algorithms in both cases produce actionable insights for the caregiver in the form of visualizations/recommendations on the communication device of the caregiver, patient, doctor, and/or any other relevant party.

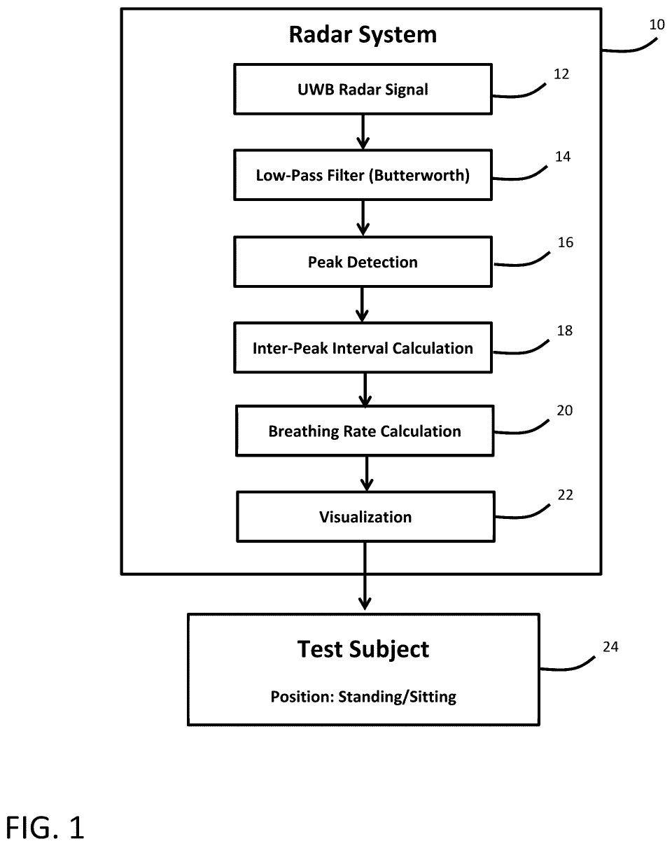

Referring now to FIG. 1, shown is a flowchart of signal processing performed in some embodiments on signals from radar system 10, such as an ultra-wide band (UWB) radar system sending UWB signals and receiving return (reflected) UWB signals 12, on which signal processing is performed. Such signal processing may include any or all of the following:

1. Apply a low-pass filter to remove high-frequency noise from the UWB return signal 14. This can be done using a Butterworth filter or the like, for example. The Butterworth low-pass filter is designed to have a maximally flat frequency response in the passband, ensuring no ripples, and an optimal roll-off rate in the stopband. The mathematical representation of the Butterworth filter is its transfer function in the Laplace domain:

$$\\ H ( s ) = \frac{1}{1 + {( \frac{s}{ω\_c} )}^{2 n}}$$

where H(s) is the transfer function, s is the complex frequency variable, ω_c is the cutoff angular frequency (ω_c=2π×cutoff frequency), and n is the order of the filter. The filter provides a smooth transition between the passband and the stopband with a roll-off rate of 20 n dB/decade.

2. Use a peak detection algorithm to identify peaks in the filtered signal 16. Peaks correspond to inhalation events. Each peak may be considered to demarcate a single breathing cycle that includes an exhalation event followed by an inhalation event. In embodiments, identifying peaks may entail scanning through the filtered signal to find local maxima in the signal. A point in the signal is identified as a peak if its amplitude is higher than its immediate neighbors. In the following pseudo code, the ‘find_peaks’ function also ensures that each detected peak is at least a certain number of samples apart from the previous peak (specified by the ‘distance’ parameter), reducing the likelihood of false positives in peak detection.

3. Compute the time intervals between consecutive peaks to get inter-peak intervals 18. The time intervals between successive peaks, which represent the time between two consecutive breaths of an individual's breathing cycle, are calculated using the following formula:

$$\\ InterPeak Interval = \frac{( Index of Peak ( i + 1 ) - Index of Peak ( i ) )}{fs}$$

where ‘Index of Peak (i)’ is the sample index of the ith peak, and ‘fs’ is the sampling frequency in Hz. The inter-peak intervals, measured in seconds, indicate the duration of one complete breathing cycle.

4. Calculate a breathing rate (BR) 20 by taking the reciprocal of the average time interval between peaks:

Breathing Rate=1/Mean of InterPeak Intervals

This provides the average rate of breathing in breaths per second. It is an essential metric in monitoring respiratory patterns.

5. A visual representation (“visual”) involving some or all of the foregoing is generated 22. The following example pseudo code includes a visualization part where the original signal, the filtered signal, and the detected peaks are plotted. Such a visual representation is crucial for understanding the signal processing steps, verifying the accuracy of peak detection, and illustrating the correlation between the detected peaks and the breathing cycles in the signal. The visual may include a printed graph as output, or an electronic graph presented on a screen, such as a screen on a tablet computer or a mobile phone, for example.

Additional Analysis may be performed to promote accuracy in the results of the foregoing. For example, further time-domain analysis may be performed using techniques like autocorrelation or frequency-domain analysis using fast Fourier transforms (FFT) to refine the breathing rate estimation. In general, autocorrelation can help identify periodic patterns in the signal, while FFT can reveal dominant frequencies.

The following pseudo code is illustrative of the foregoing.

```
import numpy as np
import matplotlib.pyplot as plt
from scipy.signal import butter, Ifilter, find_peaks
def butter_lowpass_filter(data, cutoff, fs, order=4):
  b, a = butter_lowpass(cutoff, fs, order=order)
  y = Ifilter(b, a, data)
  return y
def find_breathing_rate(signal, fs):
  # Step 1: Apply low-pass filter
  cutoff_frequency = 0.5 # Adjust as needed
  filtered_signal = butter_lowpass_filter(signal, cutoff_frequency, fs)
  # Step 2: Peak detection
  peaks, _ = find_peaks(filtered_signal, distance=fs/2) # Adjust distance based on the signal
  # Step 3: Calculate inter-peak intervals
  inter_peak_intervals = np.diff(peaks) / fs
  # Step 4: Calculate breathing rate
  breathing_rate = 1 / np.mean(inter_peak_intervals)
  # Optional: Plotting for visualization
  plt.figure(figsize=(10, 6))
  plt.plot(signal, label=‘Original Signal’)
  plt.plot(peaks, signal[peaks], ‘ro’, label=‘Peaks (Breaths)’)
  plt.plot(filtered_signal, label=‘Filtered Signal’)
  plt.xlabel(‘Sample’)
  plt.ylabel(‘Amplitude’)
  plt.title(‘UWB Radar Signal and Breathing Rate Estimation’)
  plt.legend( )
  plt.show( )
  return breathing_rate
  # Example usage
  if_{——}name_{——}== “_{——}main”:
  # Generate a sample UWB radar signal with breathing rate
  fs = 1000 # Sample rate in Hz
  t = np.arange(0, 10, 1/fs)
  uwb_radar_signal = np.sin(2 * np.pi * 0.2 * t) + 0.5 * np.random.normal(size=len(t))
  # Extract breathing rate
  breathing_rate = find_breathing_rate(uwb_radar_signal, fs)
  print(f“Estimated Breathing Rate: {breathing_rate :. 2f} breaths per second”)
```

An illustrative example signal may be provided as a synthetic UWB radar signal, such as for testing the efficacy of the foregoing, mathematically represented as:

UWB Radar Signal=sin(2πft)+Noise

where sin(2πft) is a sine wave mimicking a breathing pattern with frequency ‘f’, and ‘Noise’ is random Gaussian noise added to simulate real-world signal conditions. This synthetic signal can help demonstrate the effectiveness of the algorithm in extracting the breathing rate from a noisy periodic signal.

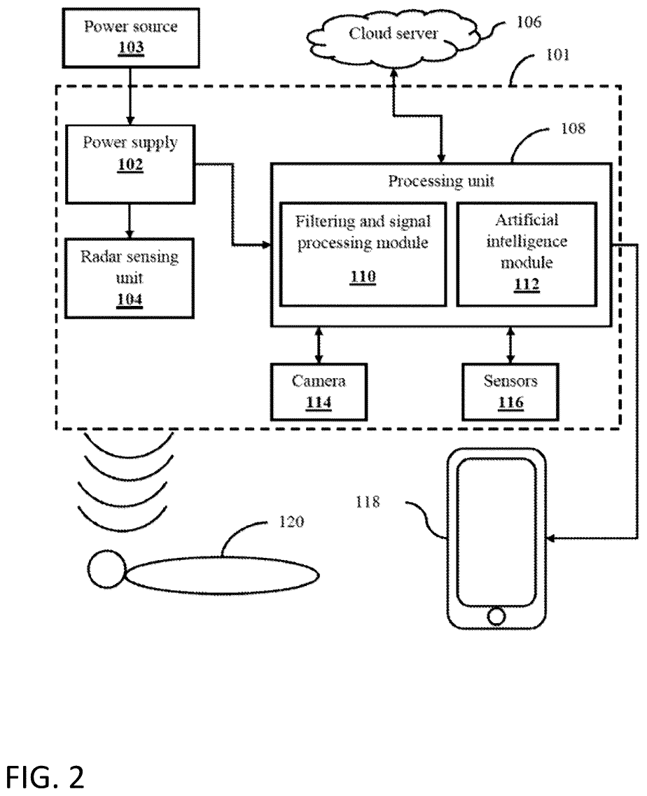

Referring now to FIG. 2, a system diagram illustrating a non-invasive, non-contact health monitoring environment is illustratively depicted, in accordance with an embodiment of the present invention.

According to an embodiment, the system diagram includes a surface mounted device 101, a monitoring device 118, a cloud server 106, and a power source 103, monitor a patient under observation 120. According to an embodiment, the surface mounted device 101 includes a power supply module 102, a radar sensing unit 104, a processing unit 108, a filtering and signaling module 110, an artificial intelligence module 112, a camera 114, and a sensor module 116. The power supply 102 receives external power from an external power source 103, such as an AC power adapter.

According to an embodiment, the patient under observation 120 is a subject whose respiration and motion patterns are monitored using the sensing unit 104.

According to an embodiment, the power supply 102 provides power to the radar sensing unit 104 for detecting the respiration and sleep pattern and also to the processing unit 108 for processing the information required for analyzing the patients. The power supply 102 also includes an alternative power source such as a backup Battery which will be used as an alternate power source in the event of power outages.

The radar sensing unit 104 is part of the wall-mounted device 101 for receiving the respiration and motion signals of the patient under observation 120. The radar sensing unit 104 is preferably placed in close proximity to the patient under observation 120. According to an embodiment of the present invention, the distance is not more than 5 m, to ensure that the patient under observation 120 is in a 60-degree cone of the sensing system. Further, the radar sensing unit 104 includes a number of components that has the capability to filter the primary noise while receiving the signals of respiration and motion.

The radar sensing unit 104 sends the received physiological data to the processing unit 108. According to an embodiment of the present invention, the processing unit 108 is a part of the wall (or table) mounted device 101. According to an embodiment of the present invention, the processing unit 108 is located inside the wall (or table) mounted device. The processing unit 108 processes the received physiological data from the radar sensing unit 104. The processing unit 108 includes the filtering and signal processing module 110 and the artificial intelligence module 112. The filtering and signal processing module 110 filters the unnecessary signals transmitted by the radar sensing unit 104 and processes the signals to derive the pattern of respiration and motion. Further, the combination of the derived pattern is combined and is compared with a set of predetermined patterns. Further, according to an embodiment, the artificial intelligence module 112 uses artificial intelligence algorithms to derive patterns and recognize abnormalities in the condition of the patient under observation 120.

The processing unit 108 receives sensing data from the camera 114 and the sensor module 116. The camera 114 is configured for providing visuals of the patient under observation. The sensor module 116 includes one or more sensors that determine temperature of the patient, sound, air quality, humidity and temperature of the room. The examples of the sensors used in the sensor module 116 include, but are not limited to, a temperature sensor, a humidity sensor, an acoustic sensor, and the like. According to an embodiment, the sensor module 116 includes an ultrasound reader, an ultra-wide band radar wave sensor, an air quality sensor, a camera, a video recorder, a microphone 225, and/or any other suitable sensor. According to an embodiment, the camera takes images and/or videos of the patient and sends the images and/or video to a secondary device, such as a mobile electronic device 118.

According to an embodiment, the cloud server 106 stores data collected from the wall (or table) mounted device and related information of the patients. According to an embodiment, the cloud server 106 is configured such that it is capable of performing processing of the data to analyze and derive information about the patients under observation. According to an embodiment of the present invention, the processing unit also transmits the processed signals to the cloud server 106 for future reference.

According to an embodiment of the present invention, whenever an abnormality in the patient's condition is detected, an alarm is raised and a notification is sent to an authorized caregiver through the monitoring device 118. The examples of the monitoring device 118 include, but are not limited to a smartphone, a laptop, a wearable device, a smart television, a tablet computer, and the like. The examples of the authorized caregivers include, but are not limited to a concerned physician, a family member, a nurse, a hospital helpline number, a friend, a third-party professional, and the like. The notification for the caregiver is sent in a plurality of ways, including but not limited to an SMS, a call, an alert warning through a mobile application, an internet messaging, and the like. According to an embodiment, the notification is sent if a pre-determined health condition is met.

According to an embodiment of the present invention, the processing unit 108 combines the respiration and motion patterns of the patients under observation to determine the early signs of a plurality of diseases and syndromes. The examples of the diseases and syndromes detected include, but are not limited to sleep apnea, epilepsy, insomnia, heart rate, seizure, cardiac arrest, sudden infant death syndrome (SIDS), respiration patterns, movement patterns, and the like.

According to an embodiment, the notification for the caregiver is sent by means of different communication networks. The examples of the communication network include, but are not limited to the Internet, an intranet, a radio-frequency network, telephonic network, a local area network (LAN), a wide area network (WAN), a proximity network such as Bluetooth, Wi-Fi, ZigBee, Bluetooth Low Energy (BLE), and the like.

According to an embodiment, the present system may incorporate presence detection, determining, e.g., intrusion detection, the presence of one or more individuals in a room, and/or any other suitable presence detection. According to an embodiment, the present system may incorporate medical technology and be configured for use in hospitals and/or other medical locations for use in medical diagnoses. Data may be sent to a nurse's (or other medical professional) station, where the nurse (or other medical professional) could be monitoring the vital signs of one or more patients. According to an embodiment, the analyzed data may be sent to a doctor (or other medical professional) in the form of a comprehensive health report.

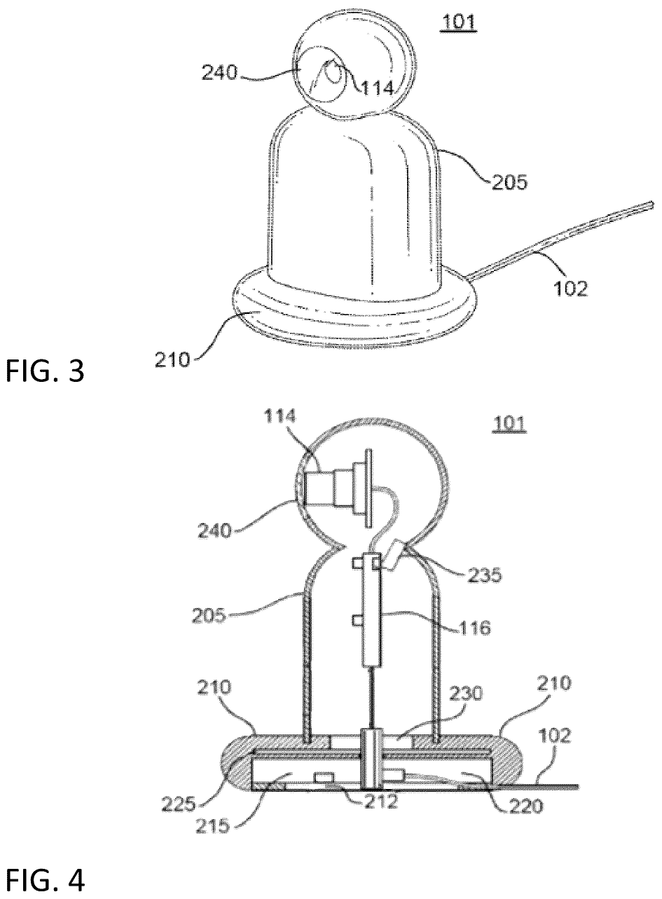

According to an embodiment, the present system may be used in the diagnosis of sleep apnea or other sleep related conditions. According to an embodiment, the present system may be used in seizure detection, epilepsy monitoring, monitoring for asthma and/or other respiratory issues, and/or the detection and/or monitoring of any other suitable ailments. Referring now to FIGS. 3-4, a perspective view (FIG. 3) and a sectional side view (FIG. 4) of an example mounted device 101 are illustratively depicted, in accordance with an embodiment of the present invention.

As shown in the illustrated embodiment, the mounted device 101 includes an outer housing 205. The outer housing 205 includes a base section 210. The base section 210 may include one or more securing mechanisms 212 configured to secure the mounted device 101 to a surface of a wall or table or the like.

In the illustrated embodiment, the mounted device 101 further includes a processor 215, a memory 220, a microphone 225, a speaker 230, an orientation mechanism 235, one or more sensors 116, and/or a camera 114. It is noted, however, that the mounted device 101 may further include any other suitable elements for the performance of the mounted device 101. According to an embodiment, a transparent covering 240 is located in front of the lens of the camera 116. According to an embodiment, the mounted device 101 includes a power supply 102, which may be a conventional electricity mains or a rechargeable or replaceable battery.

According to an embodiment, the orientation mechanism 235 aids in determining the position of a wall (or table) mounted device 101.

According to an embodiment, the processor 215 may perform one or more of the functions of a wall (or table) mounted device 101 as described herein.

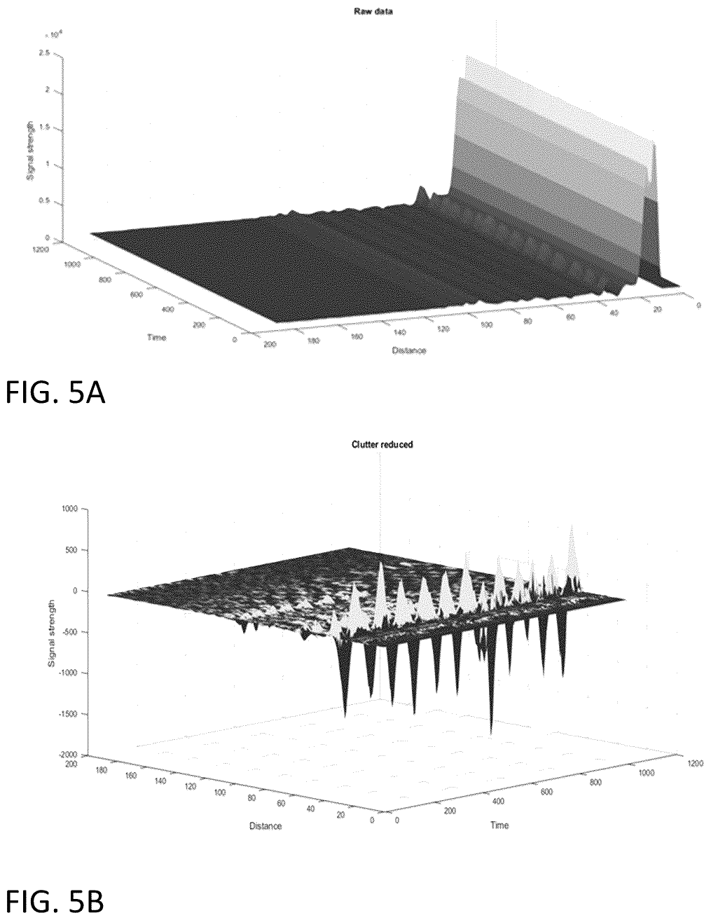

FIGS. 5A and 5B illustrate an example contactless sensing scenario. FIG. 5A shows a graph of raw data of a human being breathing at a distance of about 9 meters from a contactless sensor for 60 seconds. Such signals may be collected from a moving or a non-moving subject. FIG. 5B shows data after filtering out noise in order to obtain a clear signal, from which health information may be derived. The signals obtained are collected over a distance between 0 to 9 meters from the sensor, at a rate of 20 samples per second. Digital signal processing algorithms and artificial intelligence algorithms may be used to convert the samples to respiratory rate and movement data.

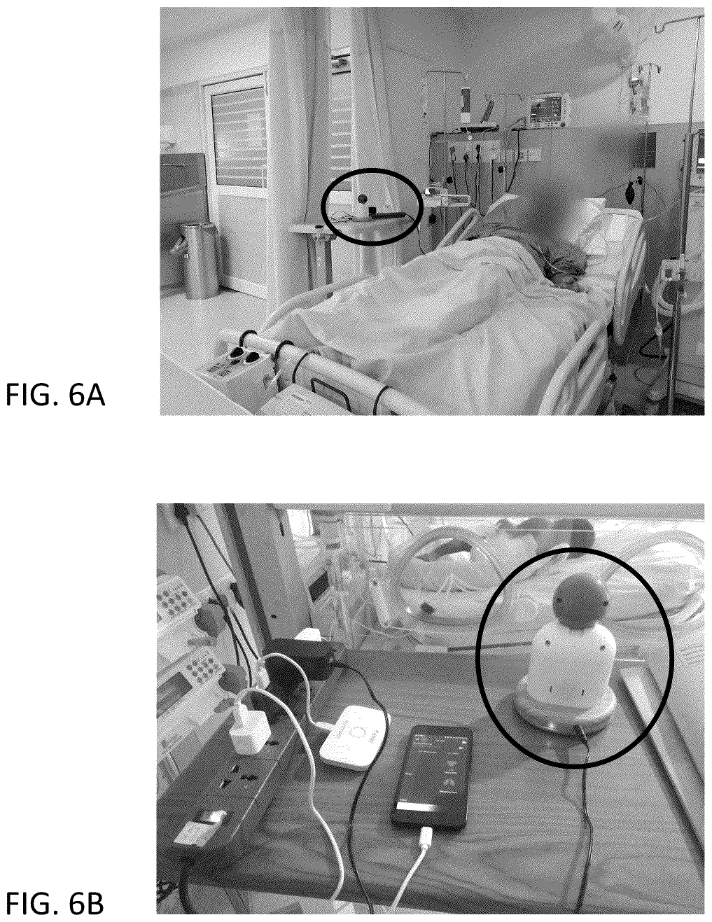

FIG. 6A illustrates placement example embodiment identified with an encircling oval., disposed near a bed in which a subject is at rest. FIG. 6B is a close-up view of the sensor setup. As shown, the sensing unit is identified with an encircling oval, and is disposed on a table by the bedside.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for monitoring a patient's health, the system comprising a monitoring device that includes:
a camera for receiving visual data, determining motion of a subject, and detecting presence of one or more non-subjects in the visual field, the camera disposed within a transparent covering;
one or more sensors including at least one of:
an infrared (IR) sensor for motion and presence detection; and
an ultra wide-band (UWB) radar sensor;
a microphone;
a speaker;
a main controller printed circuit board (PCB);
a processor operatively coupled via the PCB to at least one of the camera, the sensor(s), the microphone, and the speaker components of the monitoring device, the processor executing software that implements a signal filter having a transfer function:
$$\\ H ( s ) = \frac{1}{1 + {( \frac{s}{ω\_c} )}^{2 n}}$$
and a housing containing the PCB, the processor, the camera, the sensor(s), the microphone, and the speaker components of the monitoring device.

2. The system of claim 1, wherein the housing is mounted on a surface of a wall or table.

3. The system of claim 1, wherein the IR sensor is for use in the absence of visible light.

4. The system of claim 1, wherein the microphone is for use in situations where the subject is not mobile or is a minor of an age that entails the use of a baby monitor.

5. The system of claim 1, wherein the speaker is for use in situations where a subject is not mobile or is a minor of an age that entails use of a baby monitor.

6. The system of claim 1, wherein the UWB radar sensor includes a transceiver for transmitting and receiving electromagnetic signals, wherein the receiving signals carry position and movement data of the subject.

7. The system of claim 1, wherein at least one component of the monitoring device is operatively coupled to the main controller PCB using at least one auxiliary PCB.

8. The system of claim 1, wherein at least one of the radar sensor, the IR sensor, the camera, and the microphone are configured to provide signal data in real time from which health monitoring information of the subject is determined, the health monitoring information including a respiration rate and one or more motion patterns of a subject.

9. The system of claim 8, wherein the subject is not more than 5 meters away from the front of the radar sensor, wherein the radar sensor provides its signal data through non-physical contact using UWB radar signals.

10. The system of claim 8, wherein the radar sensor is comprised in a radar unit that further includes a transceiver chip set that receives and sends the signal data.

11. The system of claim 10, wherein the monitoring device continuously uploads raw signal data to a cloud server where the signal data is processed and analyzed to provide information to a user in real time in readable form via a mobile app.

12. The system of claim 11, wherein the data processing involves digital signal processing (DSP) to apply filters, improve signal to noise ratio, extract relevant information, and perform a machine learning/artificial intelligence (AI) algorithm to detect patterns in the data and provide insights on health and wellbeing based on the respiration rate and motion patterns.

13. The system of claim 12, further comprising an alert system to send warnings in the event of a deviation from one or more health monitoring information ranges, wherein health monitoring information values within the ranges are indicative of the subject's good health.

14. The system of claim 13, wherein the health monitoring information ranges are determined by default, by manual selection, or by automatic calculation based on the signal data.

15. The system of claim 1, wherein data of signals to or from at least one of the radar sensor, the IR sensor, the camera, and the microphone, are recorded in real time in a computing cloud.

16. A method for monitoring a patient's health, comprising:
obtaining non-contact health monitoring signal data of a subject by a health monitoring device that includes a camera, an IR sensor, a radar sensor, a microphone, and a speaker, the health monitoring signal data including:
visual signal data from the camera, for determining motion of a subject, and detecting presence of one or more non-subjects in the visual field;
IR signal data from the IR sensor, for motion and presence detection;
radar signal data from the radar sensor, for range detection;
sound signal data from the microphone; and
noise signal data from the speaker;
wherein at least a portion of the signal data from at least one of the camera, the IR sensor, the radar sensor, the microphone, and the speaker is filtered by a signal data filter having a transfer function:
$$\\ H ( s ) = \frac{1}{1 + {( \frac{s}{ω\_c} )}^{2 n}}$$

17. The method of claim 16, further comprising:
continuously transmitting the obtained signal data in real time to a computing cloud where the signal data is received, processed, and analyzed to provide health monitoring information of the subject, to a user in real time in a readable form presented via a mobile app,
wherein the health monitoring information includes a respiration rate and one or more motion patterns, determined using digital signal processing (DSP) to apply filters, improve signal to noise ratio, extract relevant information, and perform at least one machine learning/artificial intelligence (AI) algorithm.

18. The method of claim 17, wherein the respiration rate calculation comprises:
applying a low-pass filter to remove high frequency noise from radar return signal data;
identifying local maxima in the filtered signal data as the peaks of inhalation events;
computing a time interval between adjacent peaks;
calculating a breathing rate (BR) as:
BR=1/(Average Inter-Peak Interval)
visually presenting at least one of:
unfiltered signal data;
low-pass filtered signal data;
a real-time sequence of inhalation peaks;
an average inter-peak interval; and
a breathing rate calculated in real time.

19. The method of claim 17, further comprising sending a warning in the event of a deviation from one or more health monitoring information ranges,
wherein health monitoring information values within the ranges are indicative of the subject's good health; and
wherein the health monitoring information ranges are determined by default, by manual selection, or by automatic calculation based on the signal data.

20. The method of claim 18, wherein data of signals to or from at least one of the radar sensor, the IR sensor, the camera, and the microphone, are recorded in a computing cloud in real time.
